(19) United States
(12) Patent Application Publication
(10) Pub. No.: US 2024/0290478 A1
(43) Pub. Date: Aug. 29, 2024

(54) SYSTEM AND METHOD FOR OPERATING ROOM WORKFLOW IMPROVEMENT LEVERAGING RTLS, HIS, AND EMR DATA SYSTEMS

(71) Applicant: Allen Medical Systems, Inc., Batesville, IN (US)

(72) Inventors: Jack Barney Sing, Batesville, IN (US); Stefano Furgoni, Porto Mantovano (IT); Jennifer C. Jenkins, West Chester, OH (US); Heather Kooiker, Caledonia, MI (US)

(21) Appl. No.: 18/589,864

(22) Filed: Feb. 28, 2024

Related U.S. Application Data
(60) Provisional application No. 63/487,411, filed on Feb. 28, 2023.

(51) Int. Cl. G16H 40/20; G16H 10/60

(52) U.S. Cl. CPC G16H 40/20; G16H 10/60

(57) ABSTRACT

An operating room logistics system including a user device having a processor and a display. The user device is configured to: access a scheduler system to obtain operating schedules identifying operations scheduled for each operating room and identifying a patient and a surgeon for each scheduled operation; access an EMR/HIS to obtain a list of team members and items needed for each scheduled operation; accessing an RTLS to obtain locations of each item and each team member; display a schedule view display screen showing each of the scheduled operations shown for each operating room, each displayed scheduled operation being selectable; receive an input when the user selects a scheduled operation; and in response to receiving the input, display a case view display screen including: a list of items and team members needed for the selected scheduled operation along with their current location.

### CROSS-REFERENCE TO RELATED APPLICATION

This application claims priority under 35 U.S.C. § 119 (e) upon U.S. Provisional Patent Application No. 63/487,411, entitled “SYSTEM AND METHOD FOR OPERATING ROOM WORKFLOW IMPROVEMENT LEVERAGING RTLS, HIS, AND EMR DATA SYSTEMS” filed on Feb. 28, 2023, by Jack Barney Sing et al., the entire disclosure of which is incorporated herein by reference.

### FIELD OF THE DISCLOSURE

The present disclosure generally relates to a system and method for workflow management of operating rooms, and more particularly to a system and method for workflow management of surgeries scheduled in operating rooms.

### SUMMARY OF THE DISCLOSURE

According to one aspect of the present disclosure, an operating room logistics system is provided that includes a user device having a processor and a display, the user device configured to: access a scheduler system to obtain operating schedules identifying operations scheduled for each operating room and identifying a patient and a surgeon for each scheduled operation; for each scheduled operation, access an electronic medical record/hospital information system (EMR/HIS) to obtain patient information associated with the identified patient, a list of team members and a list of items needed for the scheduled operation, and surgeon information associated with the identified surgeon; access a real-time location system (RTLS) to obtain current locations of each item and each team member needed for each scheduled operation; display a schedule view display screen on the display of the user device showing the operating room schedules with each of the scheduled operations shown for each operating room, each displayed scheduled operation being selectable by a user; receive an input on the user device when the user selects a scheduled operation; and in response to receiving the input, display a case view display screen on the display of the user device including: a list of items needed for the selected scheduled operation along with a current location of each of the listed items as retrieved from the RTLS; and a list of team members for the selected scheduled operation and a current location of each of the listed team members as retrieved from the RTLS.

According to another aspect of the present disclosure, a method is provided of gathering and displaying information pertaining to operating room logistics, the method including: providing a user device in communication with a scheduler system, an EMR/HIS system, and an RTLS system; accessing the scheduler system using the user device to obtain operating schedules identifying operations scheduled for each operating room and identifying a patient and a surgeon for each scheduled operation; for each scheduled operation, accessing the EMR/HIS using the user device to obtain patient information associated with the identified patient, a list of team members and a list of items needed for the scheduled operation, and surgeon information associated with the identified surgeon; accessing the RTLS using the user device to obtain current locations of each item and each team member needed for each scheduled operation; displaying a schedule view display screen on the user device showing the operating room schedules with each of the scheduled operations shown for each operating room, each displayed scheduled operation being selectable by a user; receiving an input on the user device when the user selects a scheduled operation; and in response to receiving the input, displaying a case view display screen on the user device including: a list of items needed for the selected scheduled operation along with a current location of each of the listed items; and a list of team members for the selected scheduled operation and a current location of each of the listed team members.

According to another aspect of the present disclosure, a non-transitory computer-readable medium is provided having stored thereon instructions that, when executed by a processor in a user device, cause the user device to: access a scheduler system to obtain operating schedules identifying operations scheduled for each operating room and identifying a patient and a surgeon for each scheduled operation; for each scheduled operation, access an EMR/HIS to obtain patient information associated with the identified patient, a list of team members and a list of items needed for the scheduled operation, and surgeon information associated with the identified surgeon; access a RTLS to obtain current locations of each item and each team member needed for each scheduled operation; display a schedule view display screen on the user device showing the operating room schedules with each of the scheduled operations shown for each operating room, each displayed scheduled operation being selectable by a user; receive an input on the user device when the user selects a scheduled operation; and in response to receiving the input, display a case view display screen on the user device including: a list of items needed for the selected scheduled operation along with a current location of each of the listed items; and a list of team members for the selected scheduled operation and a current location of each of the listed team members.

These and other features, advantages, and objects of the present disclosure will be further understood and appreciated by those skilled in the art by reference to the following specification, claims, and appended drawings.

### BRIEF DESCRIPTION OF THE DRAWINGS

In the drawings:

FIG. 1 is a block diagram showing a hospital network incorporating an operating room logistics system according to one aspect of the present disclosure;

FIG. 2 is a block diagram of a user device that may be used in the operating room logistics system of FIG. 1;

FIG. 3 is a screenshot of a schedule view display screen in a day view mode;

FIG. 4 is a screenshot of a schedule view display screen in a multi-day view mode;

FIG. 5 is a screenshot of a schedule view display screen where notes associated with an open time slot in an operating room are displayed;

FIG. 6 is a screenshot of a schedule view display screen where notes associated with a scheduled operation in an operating room are displayed;

FIG. 7 is a screenshot of a schedule view display screen filtered to show only scheduled operations pertinent to a particular user;

FIG. 8 is a screenshot of a schedule view display screen filtered to show only schedule information pertinent to housekeeping;

FIG. 9 is a screenshot of a case view display screen associated with a selected scheduled operation;

FIG. 9A is an enlarged view of the list of items window as displayed in the case view display screen of FIG. 9;

FIG. 9B is an enlarged view of the OR team window as displayed in the case view display screen of FIG. 9;

FIG. 10 is a progression of screenshots of a schedule view display screen to a case view display screen of a scheduled operation selected from the schedule view display screen, and a case view display screen with a pop-up specific search window when the search function is selected for an item in a list of items shown in the case view display screen;

FIG. 11 is a screenshot of a case view display screen with a pop-up map window;

FIG. 12 is a screenshot of a case view display screen with a pop-up audio call window;

FIG. 13 is a progression of screenshots of a case view display screen and a case view display screen with a pop-up SMS message window;

FIG. 14 is a progression of screenshots of a case view display screen and a case view display screen with a pop-up map window;

FIG. 15 is a progression of screenshots of a case view display screen and a case view display screen with a pop-up audio message window;

FIG. 16 is a progression of screenshots of a case view display screen with a pop-up case note window, and two examples of schedule view display screens where notes associated with two different scheduled operations are displayed;

FIG. 17 is a progression of screenshots of a case view display screen, a search screen, and a search screen with a pop-up audio message window;

FIG. 18 is a screenshot of a search display screen; and

FIG. 19 is a flowchart showing a method of operation of the operating room logistics system.

### DETAILED DESCRIPTION

The present illustrated embodiments reside primarily in combinations of method steps and system components related to operating room logistics. Accordingly, the system components and method steps have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, like numerals in the description and drawings represent like elements.

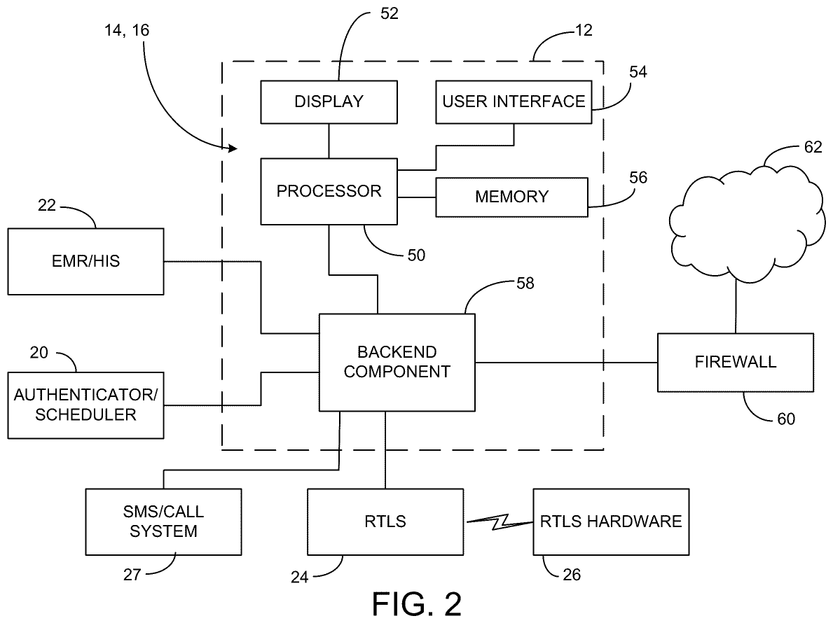

For purposes of description herein, the terms “upper,” “lower,” “right,” “left,” “vertical,” “horizontal,” and derivatives thereof, shall relate to the disclosure as oriented in FIG. 2. However, it is to be understood that the disclosure may assume various alternative orientations, except where expressly specified to the contrary. It is also to be understood that the specific structures and processes illustrated in the attached drawings and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The terms “including,” “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises a . . . ” does not, without more constraints, preclude the existence of additional identical elements in the process, method, article, or apparatus that comprises the element.

Operating room (OR) management during a typical workday is quite chaotic. A charge nurse (also known as a supervising nurse) is typically in charge of managing many ORs. There may be as many as 10-20 OR suites in a hospital. OR suites must be prepared properly, supplied for the planned operation, and staff must be allocated appropriately and in a timely manner. Any breakdown in staging, planning, or execution can cause delays and have a ripple effect in terms of efficiency, time, error elimination, and overall stress. An OR logistics system and method of OR logistics management is described below that greatly improves upon the technology within the hospital to thereby drive data to staff, thereby increasing accuracy in decision making, allocating resources, and compressing time for the OR. This compression will allow the attending staff to focus on the procedure, improving overall care and increasing system and process efficiency.

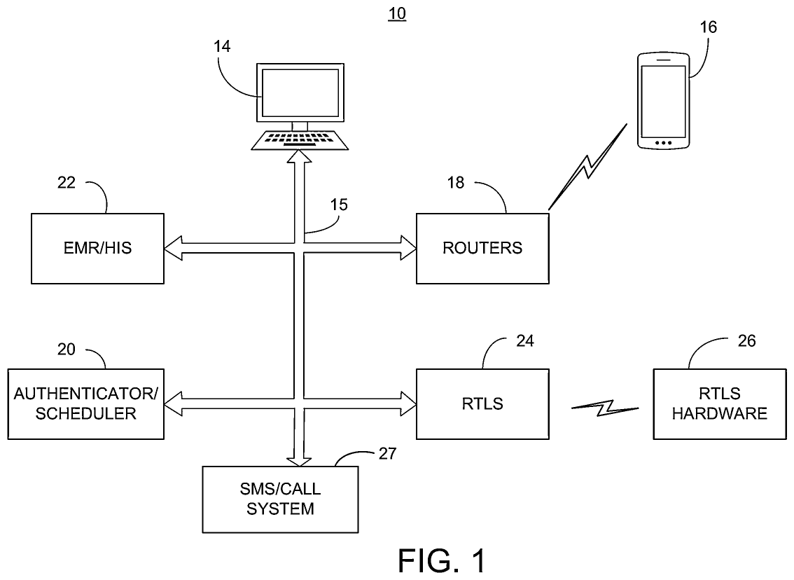

Referring to FIG. 1, reference numeral 10 generally designates a hospital network in which an operating room logistics system 12 may be implemented. As shown, the hospital network 10 may include a plurality of user devices 14 and 16 connected directly to a network bus 15 or indirectly through wireless routers 18. The hospital network 10 may also include an authenticator/scheduler system 20 (hereinafter simply referred to as “the scheduler system”), an electronic medical record/hospital information system (EMR/HIS) 22, and a real-time location system (RTLS) 24, which all may be implemented on one or more servers connected to the network bus 15. The RTLS 24 may be wirelessly connected to various RTLS hardware 26 that is associated with various items and personnel in the hospital for purposes of tracking the location of the items and personnel.

The scheduler system 20 may also be any conventional system provided the scheduler system 20 can provide accessible operating schedules identifying operations scheduled for each operating room and identifying a patient and a surgeon for each scheduled operation. Further details of the information that the operating room logistics system 12 may access from the scheduler system 20 is described further below.

The EMR/HIS 22 may also be any conventional system such as EPIC® or Cerner® provided the EMR/HIS 22 can provide accessible patient information associated with the patient identified from the scheduler system 20, a list of team members and a list of items needed for the scheduled operation, and surgeon information associated with the surgeon identified from the scheduler system 20. Further details of the information that the operating room logistics system 12 may access from the EMR/HIS 22 is described further below.

The RTLS 24 and associated RTLS hardware 26 may be any conventional system provided the RTLS 24 can provide accessible real-time location information on each of the items and team members identified from the EMR/HIS 22. Further details of the information that the operating room logistics system 12 may access from the RTLS 24 is described further below.

The SMS/Call system 27 may also be any conventional system provided it allows either phone calls or SMS text messaging.

The operation room logics system 12 is generally implemented with software executed by the user devices 14 and 16. As shown in FIG. 1, the user devices 14 and 16 may be a desktop computer 14 or a mobile smartphone 16 or any other device such as a tablet or laptop having a processor, a memory device, and a display. An example of the components of such a user device is shown in FIG. 2. As shown, the operating room logistics system 12 includes the user device 14, 16, which may include a processor 50, a display 52, a user interface 54, and memory 56. A backend component 58 connects to the authenticator/scheduler system 20, the EMR/HIS 22, the RTLS 24, a short message service (SMS)/Call system 27, and optionally, a firewall 60, which connects to a cloud-based database 62 as described further below.

The user interface 54 may be any conventional user interface and may include any one or more of: a keyboard, a mouse, a touch sensor used with the display 52 to form a touchscreen, and a microphone for receiving voice commands.

The backend component 58 may be implemented using Azure Kubernetes Service Cluster that may include an application programming interface (API) gateway (such as Azure API Management), a hypertext transfer protocol (HTTP) aggregator, a microservices (business component layer), and health checks (such as Azure monitoring). The microservices may include an integration layer such as GraphQL that: communicates with the authenticator/scheduler system 20 and the HIS/EMR 22 both using TCP/IP, TLS HL7 protocol; communicates with the RTLS server 24 using WebSockets to receive real time data; and communicates with the SMS/Call system 27 using voice over Internet protocol (VOIP).

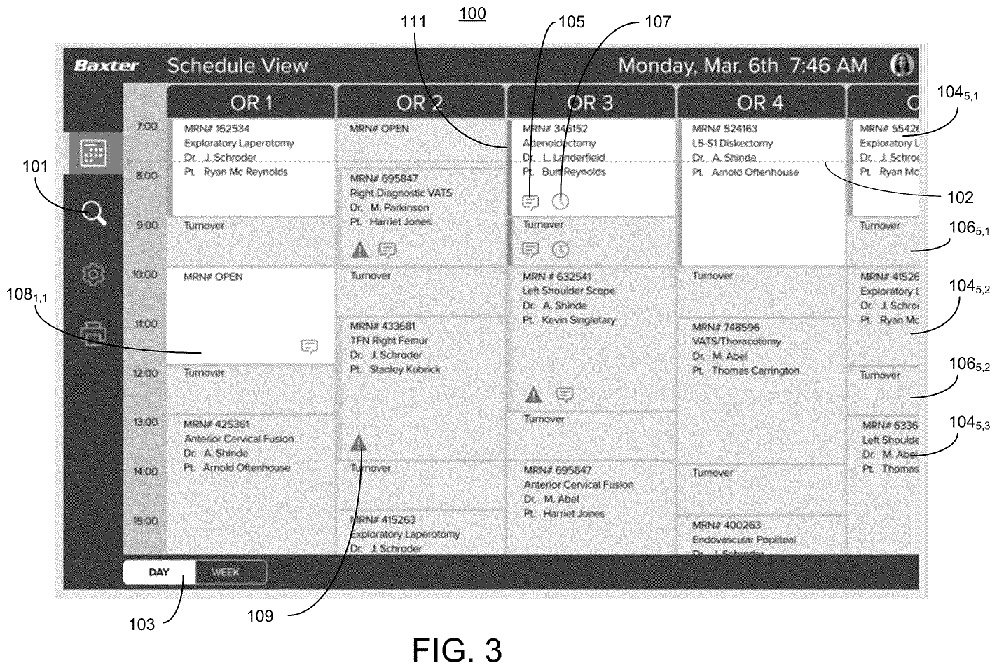

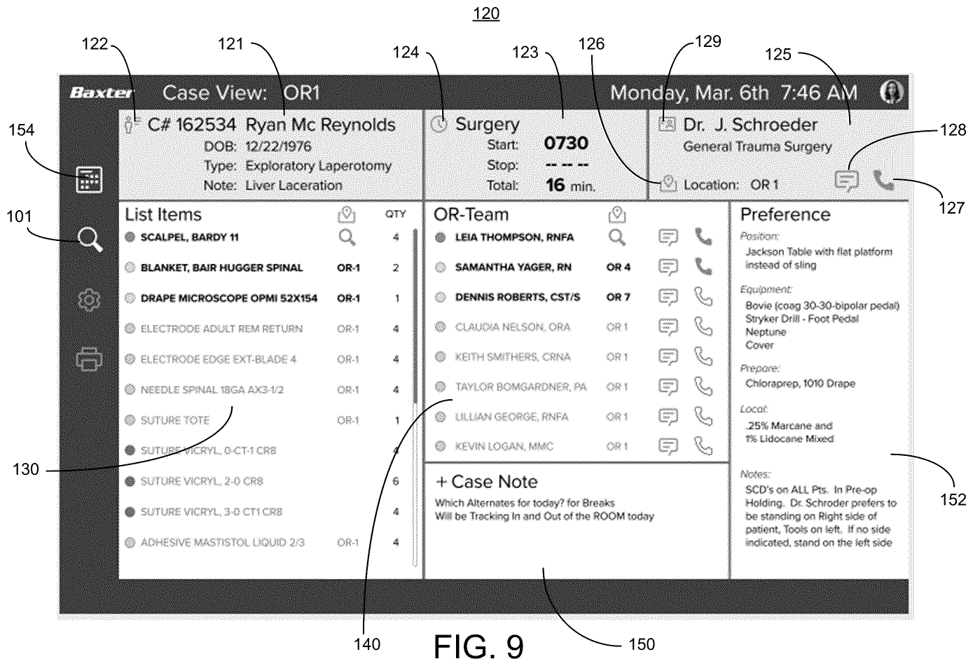

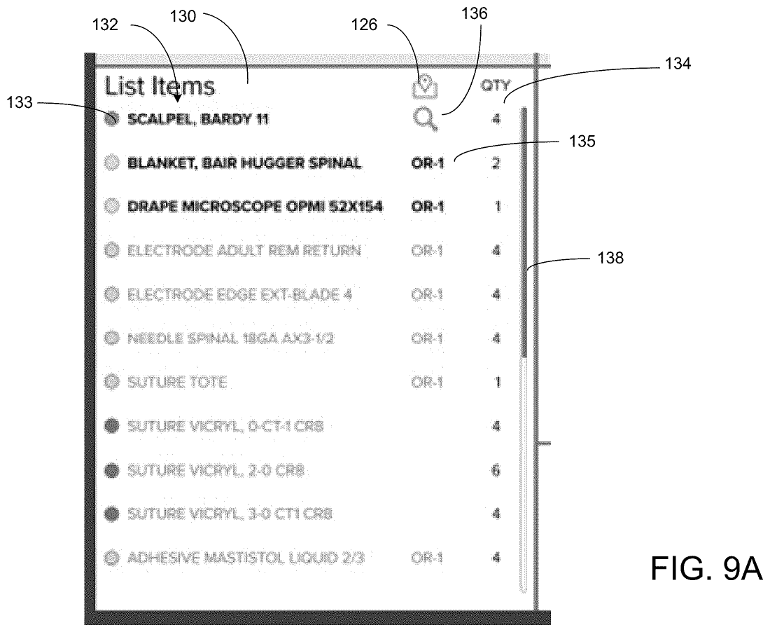

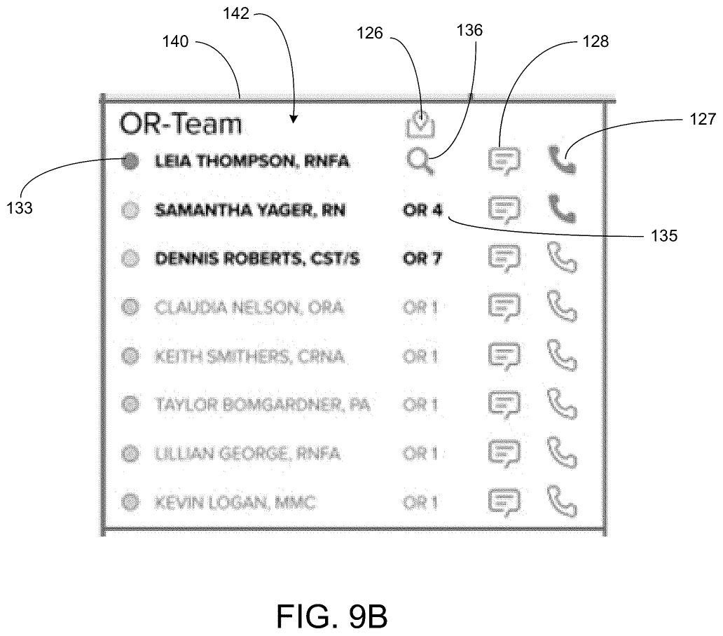

In summary, the operating room logistics system 12 includes the user device 14, 16 including at least the processor 50 and the display 52. The user device 14, 16 is configured to: access the scheduler system 20 to obtain operating schedules identifying operations scheduled for each operating room and identifying a patient and a surgeon for each scheduled operation; for each scheduled operation, access the EMR/HIS 22 to obtain patient information associated with the identified patient, a list of team members and a list of items needed for the scheduled operation, and surgeon information associated with the identified surgeon; access the RTLS 24 to obtain current locations of each item and each team member needed for each scheduled operation; display a schedule view display screen 100 (FIGS. 3-6) on the display 52 of the user device 14, 16 showing the operating room schedules with each of the scheduled operations 104_{x,y} shown for each operating room, each displayed scheduled operation 104_{x,y} being selectable by a user; receive an input on the user device 14, 16 when the user selects a scheduled operation 104_{x,y}; and in response to receiving the input, display a case view screen 120 (FIG. 9) on the display 52 of the user device 14, 16 including: a list of items 132 needed for the selected scheduled operation 104_{x,y} along with a current location 135 of each of the listed items 132; and a list of team members 142 for the selected scheduled operation 104_{x,y} and a current location 135 of each of the listed team members 142.

As noted above, the user device 14, 16 may access the RTLS 24 to obtain current locations of each item and each team member. Such access may be periodic and may be performed at such a rate as to appear to the user as nearly continuously updating the locations.

By accessing different and independent systems in the hospital network 10 and organizing and displaying the accessed information in the manner described, the operating room logistics system 12 improves the technology available to those in charge. Previously, a charge nurse would at best have access to information on the scheduler system or the EMR/HIS, but would have to access these systems separately and would have to separately enter any search criteria. The operating room logistics system 12 automatically pulls relevant data pertaining to each scheduled operation 104_{x,y} from multiple independent systems. To better understand the operation of the operating room logistics system 12, several screenshots are shown in FIGS. 3-18 and described below.

FIG. 3 is a screenshot of a schedule view display screen 100 in a day view mode where for a given day, the schedule for each operating room is displayed in a graphic form with the operating rooms (OR1, OR2, etc.) forming vertical columns extending horizontally and the time of day displayed along one side with the time increasing downwardly. A dashed horizontal line 102 may be used to indicate the current time of day. For the schedule, each operating room may include a plurality of scheduled operations 104_{x,y}. For purposes of illustration only, the reference number 104_{x,y} is used where x is an operating room number and y is the order of scheduled operations for a given day. For example, the scheduled operation 104_{3,1} is the first scheduled operation for operating room 3 (OR3). Each scheduled operation is shown graphically as a block with an upper edge at a start time and a lower edge at an expected stop time. In addition to displaying the scheduled operations, blocks of time 106_{x,y} may be displayed corresponding to “turnover.” These turnover blocks of time 106_{x,y} generally begin when a preceding scheduled operation stops and are significant for cleaning staff and prepping staff to prepare for the next scheduled operation. Further, there may be blocks of time 108_{x,y} where the operating room is open.

Each scheduled operation 104_{x,y} shown in the schedule view display screen 100 includes a case reference number, the operation to be performed, the doctor's name, and the patient's name. In addition, a note icon 105, a clock icon 107, an alert icon 109, and/or a status indicator 111 may be displayed for each scheduled operation 104_{x,y}, each turnover block of time 106_{x,y}, and each open block of time 108_{x,y}. The status indicator 111 may be in the form of a color-coded bar along one side of each block of time. The function of these icons and status indicator are discussed further below.

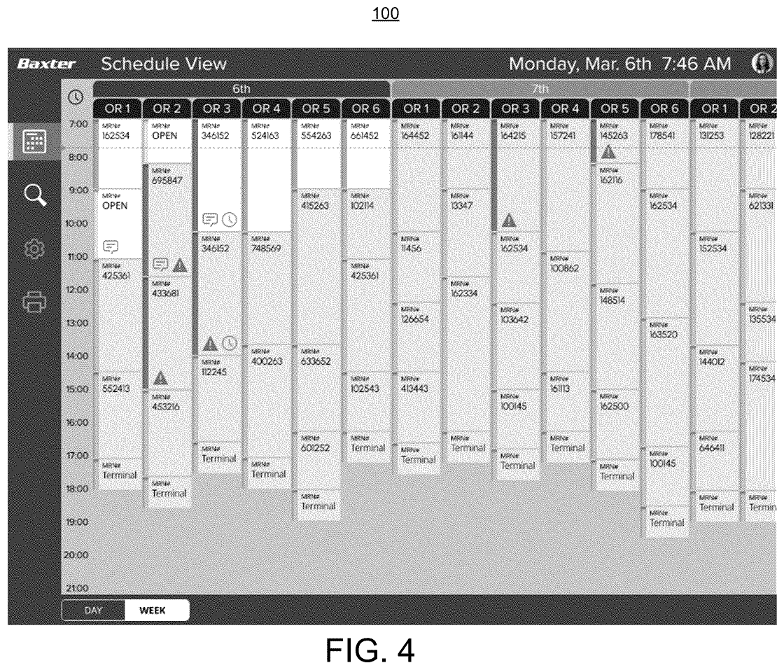

The schedule view display screen 100 may include a search icon 101 for selecting to perform a search as described further below. The schedule view display screen 100 may also include a day/week indicator 103 for indicating whether the day view mode or the week view mode is selected. If the display 52 of the user device 14, 16 is a touchscreen, the day/week indicator 103 may be used as a selector button. FIG. 4 is a screenshot of a schedule view display screen 100 in a week view mode in which schedules for multiple days are displayed at once.

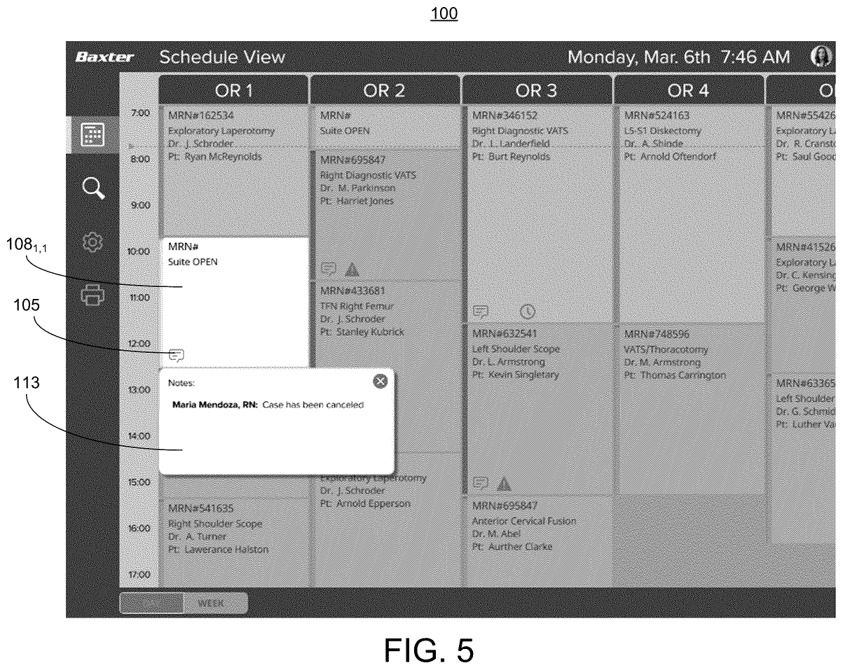

FIG. 5 is a screenshot of an example of a schedule view display screen 100 where notes associated with an open block of time 108_{1,1} in an operating room OR1 are displayed in a pop-up case note window 113 when the note icon 105 is selected.

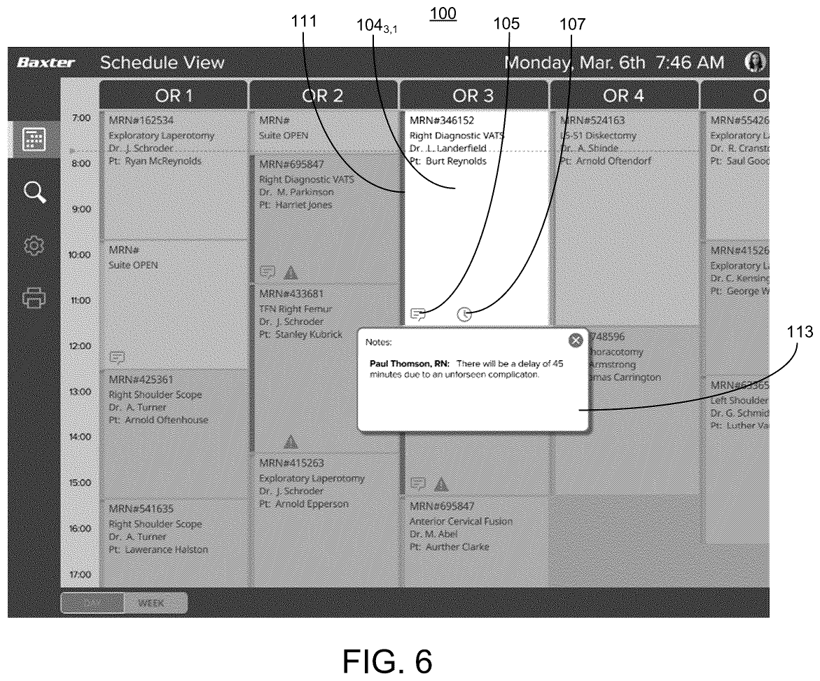

FIG. 6 is a screenshot of an example of a schedule view display screen 100 where notes associated with a scheduled operation 104_{3,1} in an operating room OR3 are displayed in a pop-up case note window 113 when the note icon 105 is selected. In this particular example, the notes show that “There will be a delay of 45 minutes due to an unforeseen complication.” This delay occurring during an operation causes the status indicator 111 to change and the clock icon 107 to appear in order to indicate that the operation is behind schedule. The status indicator 111 may change to red, for example.

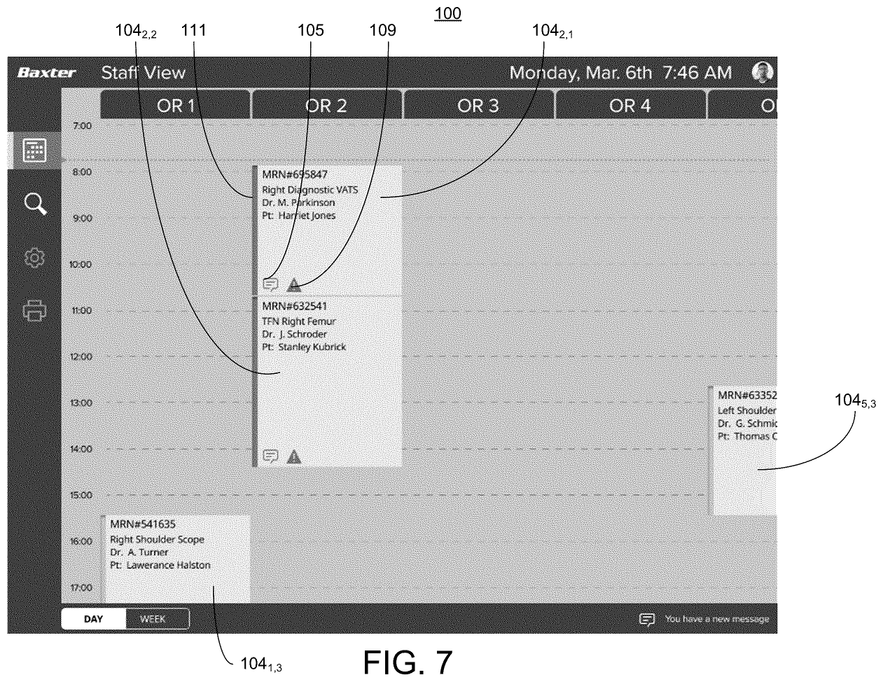

FIG. 7 is a screenshot of an example of a schedule view display screen 100 filtered to show only scheduled operations pertinent to a particular user. In this particular example, the user may be a circulating nurse who can view only the scheduled operations 104_{2,1}, 104_{2,2}, 104_{5,3}, 104_{1,3} for which they are scheduled to participate. As also shown in this example, the status indicator 111 for the first two scheduled operations 104_{2,1}, 104_{2,2} is red and an alert icon 109 is present, which indicates that something is causing a delay to the start of the first operation 104_{2,1} and consequently the second operation 104_{2,2}, which is scheduled in the same operating room OR2. The circulating nurse (or the charge nurse) may thus immediately see that a scheduling conflict may occur with the third operation 104_{5,3} in a different operating room OR5. Thus, rescheduling of staff may be done in advance rather than at the last minute.

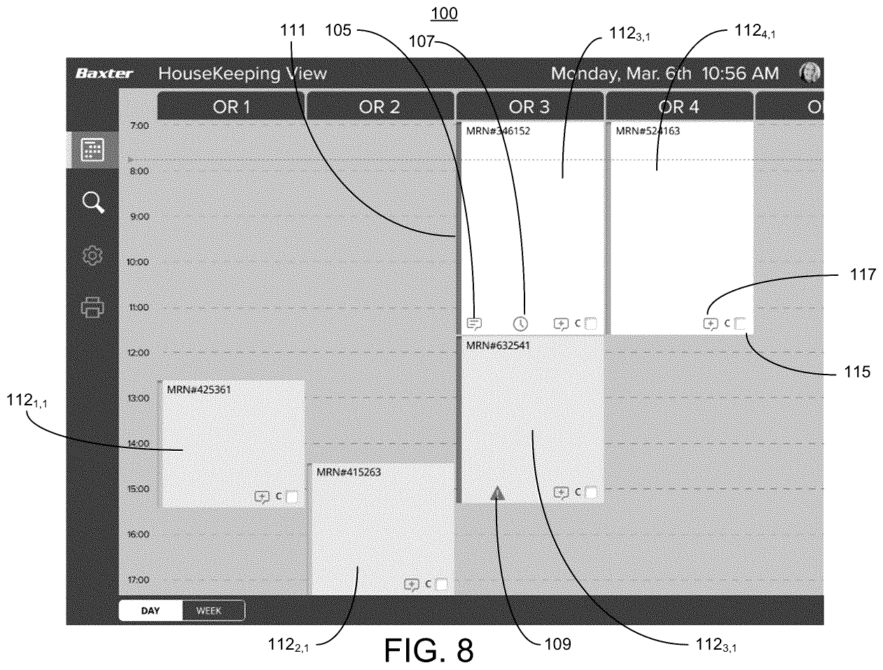

FIG. 8 is a screenshot of an example of a schedule view display screen 100 filtered to show only schedule information pertinent to housekeeping. In this example, pertinent blocks of cleaning time 112_{1,1}, 112_{2,1}, 112_{3,1}, 112_{3,2}, 112_{4,1} are shown. These blocks may correspond to any one or combination of turnover blocks of time 106_{x,y} and open blocks of time 108_{x,y}. The cleaning time blocks 112_{x,y} may include a “C” checkbox 115 that may be checked when all cleaning has been completed. Also, an add note icon 117 may be provided to allow housekeeping staff to leave notes for viewing by other staff. Note icons 105, clock icons 107, alert icons 109, and status indicators 111 may be shown that are pertinent to housekeeping staff. For example, the status indicator 111 and clock icons 107 may indicate that a prior operation is taking longer than anticipated thereby delaying the start of cleaning time available. A note icon 105 can be selected to display a note as to the reason for the delay or to display a note regarding special cleaning instructions such as to not use a particular cleaning product due to an allergy of the next patient scheduled for surgery.

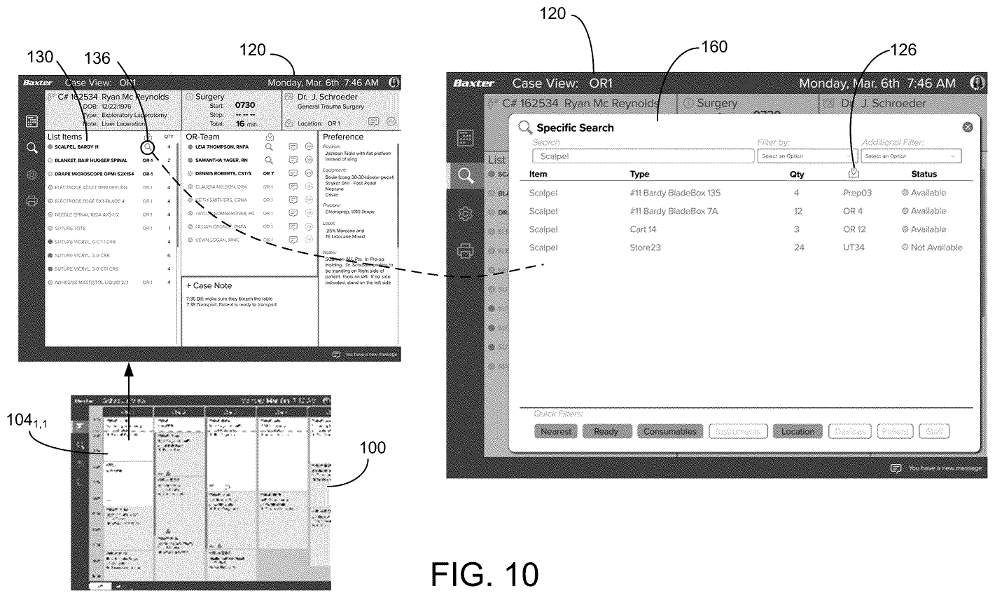

FIG. 9 is a screenshot of an example of a case view display screen 120 associated with a selected scheduled operation. FIG. 10, which is described further below, shows the progression of moving from a schedule view display screen 100 to a case view screen 120 by selection of a scheduled operation 104_{1,1} on the schedule view display screen 100. Referring to FIG. 9, and as will be described further below, the case view screen may include a patient information window 121, a surgery information window 123, a surgeon information window 125, a list of items window 130, an OR team window 140, a case note window 150, and a surgeon preference window 152. In addition, the case view display screen 120 may include the search icon 101 and a schedule view icon 154 that returns to the schedule view display screen 100.

The patient information window 121 may include patient name, date of birth (DOB), type of operation, and any pertinent notes about the patient's condition and/or allergies. This information is accessed from the EMR/HIS 22. In addition, a patient icon 122 may be displayed that, when selected, links to the patient's EMR.

The surgery information window 123 may display the scheduled start time, the time the surgery is either actually stopped or scheduled to be stopped, the total scheduled time for the surgery and/or the elapsed or remaining time for the surgery. This information is significant to decision making, although not directly related to logistic feedback. This information may be used by the operating room logistics system 12 to prompt behaviors such as messaging team members regarding case progress or other matters. For example, the user device 14, 16 may be further configured to determine if a scheduled operation 104_{x,y} is behind schedule and generates a status indicator 111 on the schedule view display screen 100 indicating that the scheduled operation 104_{x,y} is behind schedule.

The surgeon information window 125 includes information about the surgeon assigned to the scheduled operation. Such information may include the surgeon's name and type of surgeon. This information may be accessed from the EMR/HIS 22. In addition, the surgeon's current location may be displayed as obtained from the RTLS 24. A number of icons may be provided in the surgeon information window 125 including a map icon 126, a call icon 127, a message icon 128, and a profile icon 129. When selected, the map icon 126 displays a map of a portion of the hospital showing some or all of the operating rooms as well as a marker showing the surgeon's current location. When the call icon 127 is selected, a phone call pop-up window will appear allowing a phone call to be made to the surgeon. When the message icon 128 is selected, a message pop-up window will appear allowing a message to be sent to the surgeon. The message may be a text message, page message, or audio message. When the profile icon 129 is selected, surgeon's information from the EMR/HIS 22 will appear.

The case note window 150 is provided to display any notes pertinent to the scheduled operation and to allow creation of new notes. As shown relative to the schedule view display screen 100, when a note icon 105 is selected, the notes displayed in the case note window 150 appear in a pop-up case note window 113.

The surgeon preference window 152 may display surgeon information that may include surgeon preferences for set up of the operating room as well as position preference for positioning of the surgeon and surgical tools relative to patient, specific equipment preferences, local anesthesia preferences, draping preferences, and notes relating to preferences, for example. This information may be pulled from the EMR/HIS 22.

As shown in an enlarged view in FIG. 9A, the list of items window 130 may include a list of the names of all items 132 required for the scheduled operation. For each listed item 132, there may be displayed a status indicator 133, a quantity needed 134, a location 135 of the listed item 132, and if a location is not known, a search icon 136 is provided. In addition, the list of items window 130 may include a map icon 126 and a scroll bar 138 for when there are more items than can fit legibly within the list of items window 130. The status indicators 133 may change appearance (such as color) to indicate whether the item is located within the operating room where the scheduled operation is to take place, or whether the item is not located within the operating room or if it is an untracked item. The list of items and quantity required may be accessed from the EMR/HIS 22 and the location of each item may then be accessed from the RTLS 24. The operating room logistics system 12 may generate the status indicators 133 by comparing the location 135 of each item to the operating room where the scheduled operation is to take place.

As shown in an enlarged view in FIG. 9B, the OR team window 140 may include a list of the names of all team members 142 assigned for the scheduled operation. For each team member 142, there may be displayed a status indicator 133, a location 135 of the team member 142, and if a location is not known, a search icon 136 is provided. In addition, the OR team window 140 may include a map icon 126. The status indicators 133 may change appearance (such as color) to indicate whether the team member 142 is located within the operating room where the scheduled operation is to take place, or whether the team member is not located within the operating room. If the team member 142 is not in the operating room, different statuses may be indicated based on their availability. For example, a team member 142 may be unavailable if they are assigned to another scheduled operation that has been delayed. The list of team members 142 required may be accessed from the EMR/HIS 22 and the location of team member 142 may then be accessed from the RTLS 24. The operating room logistics system 12 may generate the status indicators 133 by comparing the location 135 of each team member to the operating room where the scheduled operation is to take place.

By providing the location and status of each item 132 and team member 142 on a single case view display screen 120, a charge nurse or other personnel may quickly identify whether an operating room is properly prepped for an upcoming scheduled operation or that the scheduled operation is ready to begin or is likely to be delayed. Further, if an operating room is not fully prepped, they can quickly see what items 132 or team members 142 are missing along with their location and status. As explained further below with respect to FIG. 10, if an item 132 is missing, a user can quickly locate available items 132.

As mentioned above, FIG. 10 is a progression of screenshots of a schedule view display screen 100 to a case view display screen 120 of a scheduled operation 104_{x,y} selected from the schedule view display screen 100, and a case view display screen 120 with a pop-up specific search window 160 when the search icon 136 is selected for an item 132 (e.g., scalpel) in the list of items window 130 shown in the case view display screen 120. As shown in the pop-up specific search window, a search was performed for available scalpels and four different hits were displayed that show the item name, the type of scalpel, the quantity, the location, and the availability. Note that some of the scalpels may be located but not available as they may need to be sterilized. Such a quick search feature for a missing item needed for an operation makes it much easier for the personnel managing the operating rooms to quickly search and locate available missing items.

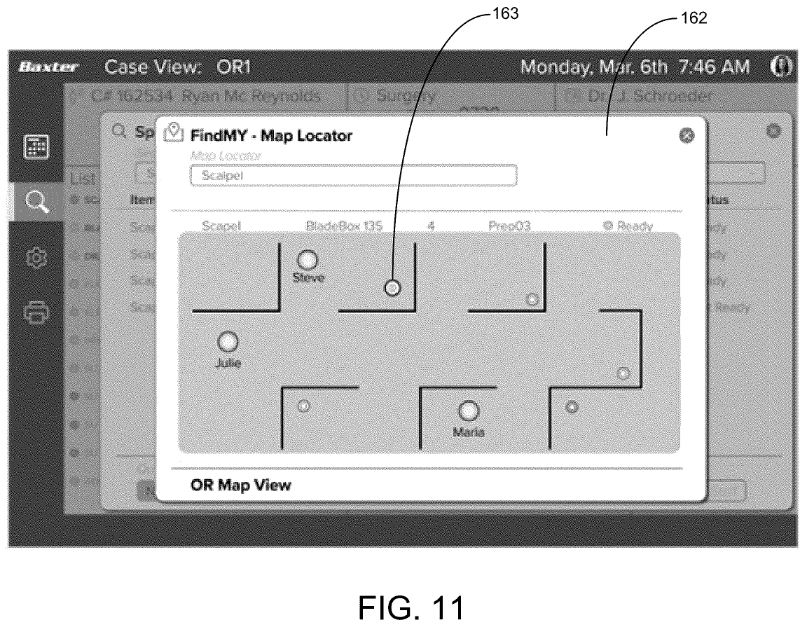

A map icon 126 may be provided in the pop-up specific search window 160. When the map icon 126 is selected, a pop-up map window 162 (FIG. 11) may be displayed. In the example shown, the location 163 of the scalpels is shown in the pop-up map window 162 thus making the available scalpels even easier to find.

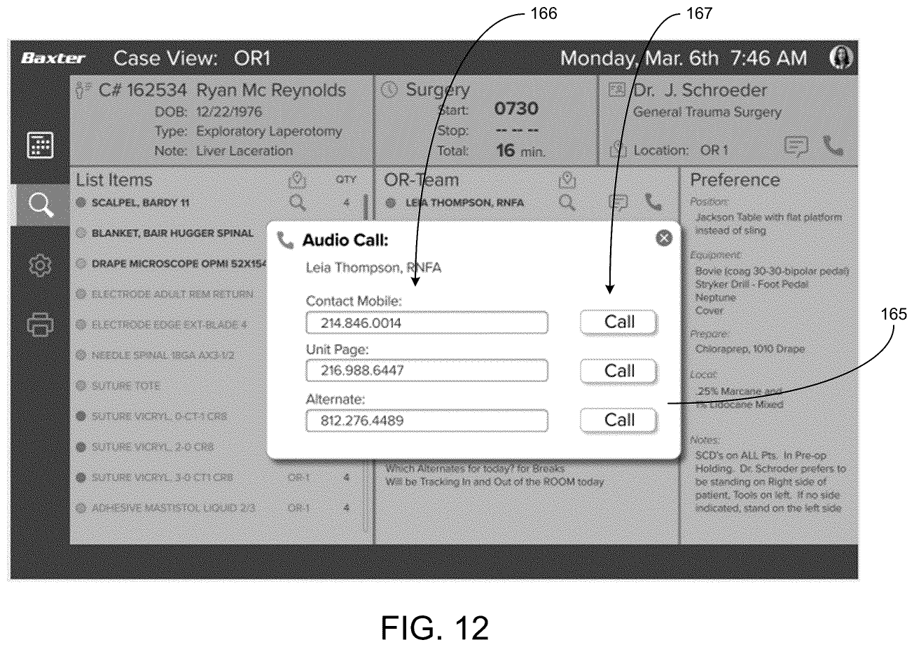

Referring back to FIG. 9B, which shows an enlarged portion of the OR-team window 140, for each team member, a call icon 127 and a message icon 128 is provided to allow quick access to communication with the team members. For example, if a team member is not available or in the required operating room, a user may select the call icon 127, which causes a pop-up audio call window 165 (FIG. 12) to be displayed. The pop-up audio call window 165 may include a list 166 of possible phone numbers (e.g., contact mobile, unit page, and/or alternate), and a call icon 167 next to each listed phone number 166. A user may initiate a call to the listed phone number 166 by selecting the associated call icon 167. This allows for quick communication with the team members of a specified scheduled operation.

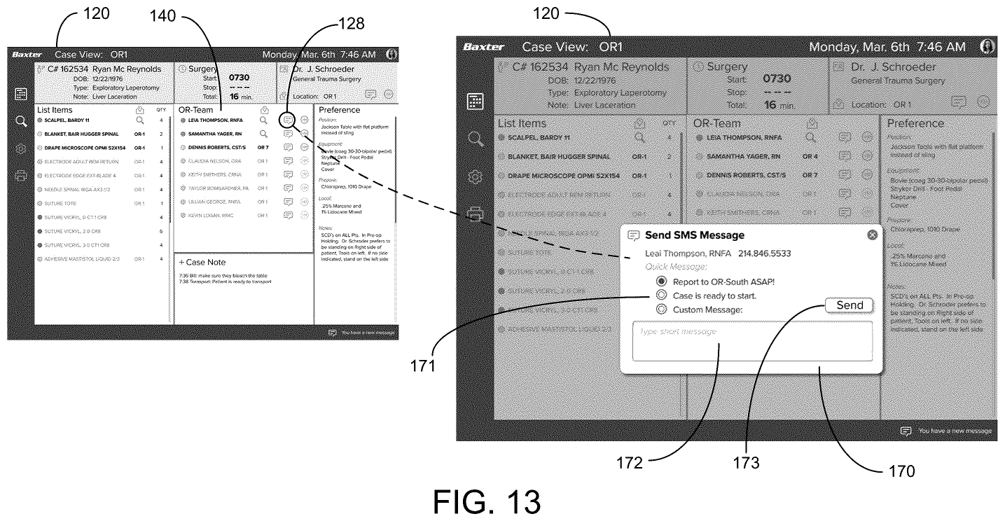

FIG. 13 is a progression of screenshots of a case view display screen 120 and a case view display screen 120 with a pop-up SMS message window 170 that results from the selection of the message icon 128 in the OR team window 140 of the case view display screen 120. The pop-up SMS message window 170 may include a message selection list 171 that lists pre-written messages and a custom message and a message writing box 172 in which a custom message may be written. The pop-up SMS message window 170 may also include a send button 173 for sending the selected message. This allows for quick communication with the team members of a specified scheduled operation.

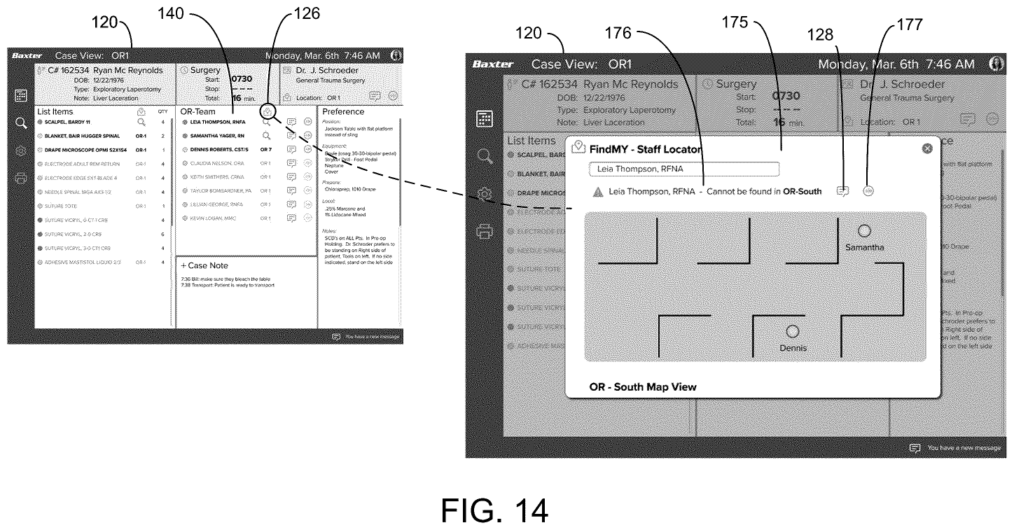

FIG. 14 is a progression of screenshots of a case view display screen 120 and a case view display screen 120 with a pop-up map window 175 that results from the selection of the map icon 126 in the OR team window 140 of the case view display screen 120. The pop-up map window 175 may include a location message 176 and a message icon 128 and an audio message icon 177 associated with the team member for which the search was conducted. This allows for quickly locating the team members of a specified scheduled operation.

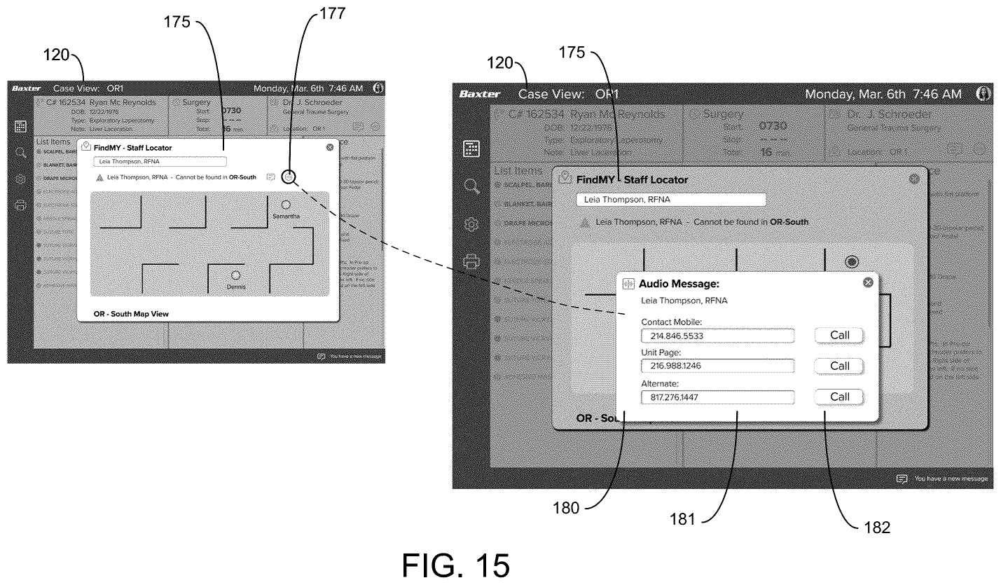

FIG. 15 is a progression of screenshots of a case view display screen 120 and a case view display screen 120 with a pop-up audio message window 180 that results when the audio message icon 177 is selected in the pop-up map window 175. The pop-up audio message window 180 may include a list 181 of possible phone numbers (e.g., contact mobile, unit page, and/or alternate), and a call icon 182 next to each listed phone number 181. A user may initiate a voicemail audio message to the listed phone number 181 by selecting the associated call icon 182. This allows for quick communication with the team members of a specified scheduled operation.

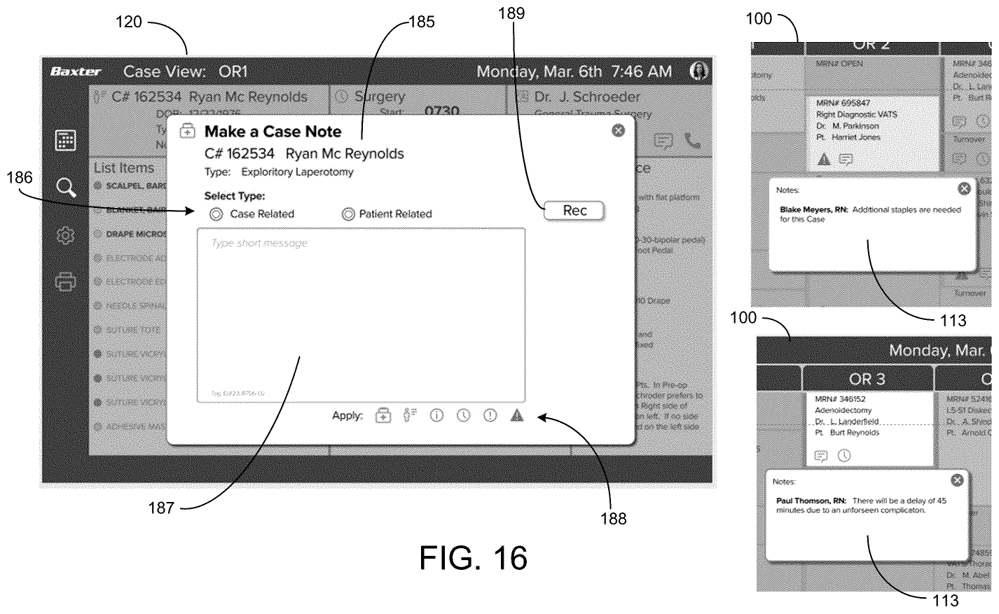

FIG. 16 is a progression of screenshots of a case view display screen 120 with a case note window 185, and two examples of schedule view display screens 100 where pop-up notes 113 associated with two different scheduled operations are displayed. The case note window 185 may include a selector menu 186 allowing a user to select whether the note is case related or patient related. Case related notes will appear on the schedule view display screens 100 as pop-up case note window 113 when a note icon 105 is selected. The case note window 185 may also include a writing box 187 in which a note may be written and may include a list of available icons 188 that may be selected and then displayed in schedule view display screen 100. Further, the case note window 185 may include a record button 189 to submit the note and record it to the case or patient records. This note generating feature is useful not only to a charge nurse, but also to OR team members to indicate that additional items are needed or that an operation is running late.

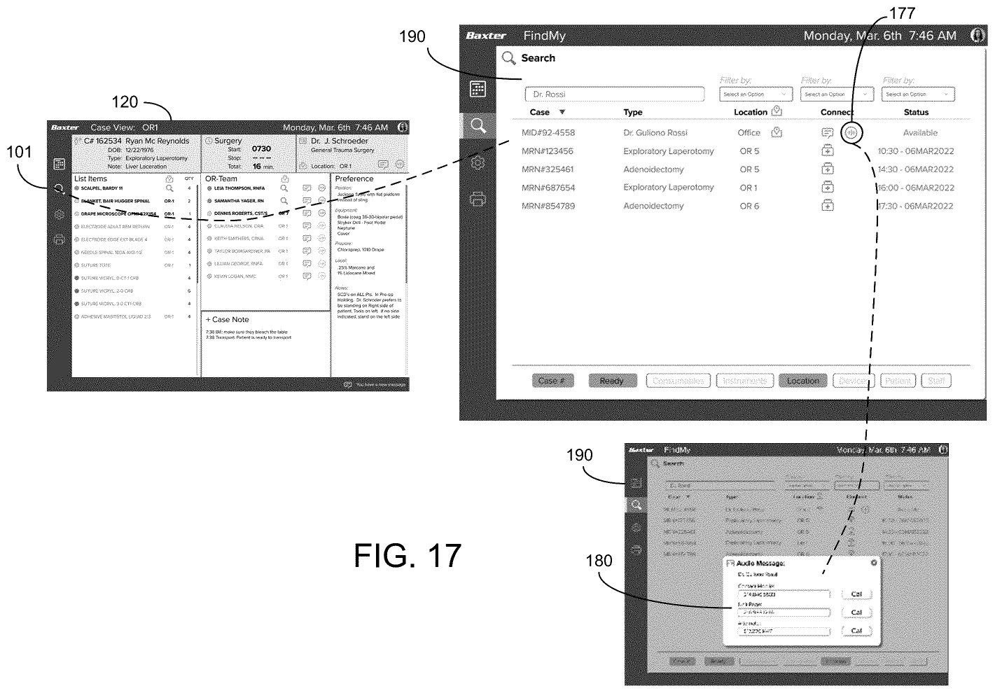

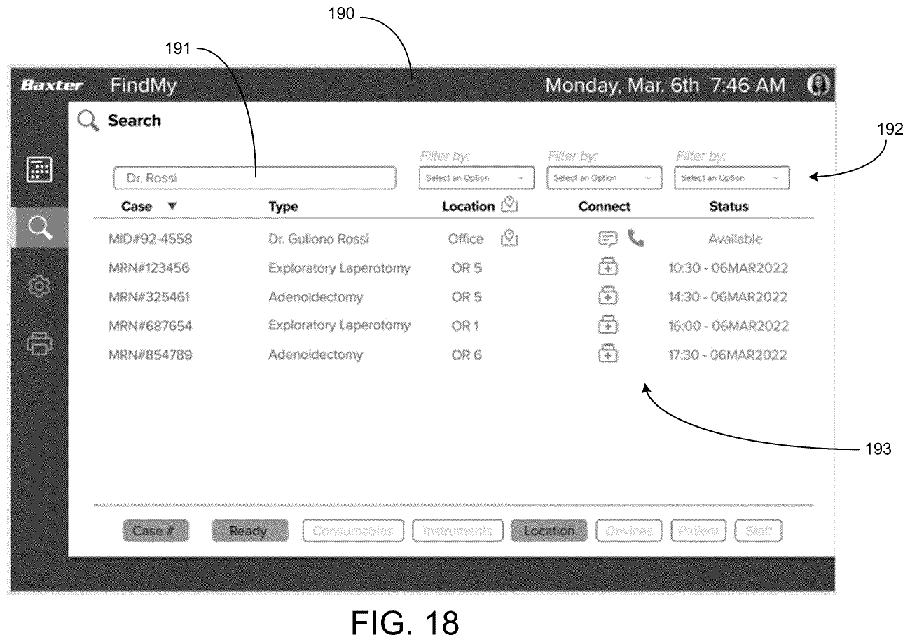

FIG. 17 is a progression of screenshots of a case view display screen 120, a search screen 190 resulting from a selection of search icon 101, and the search screen 190 with a pop-up audio message window 180 resulting from selection of the audio message icon 177 in the search screen 190. As best shown in FIG. 18, the search screen 190 may include a field 191 for entry of the item/person searched for, one or more filter pull-down menus 192, and a search result display area 193. The search result display area 193 may display various selected information pertaining to the search, such as case numbers, type, location, status, and connection and map icons.

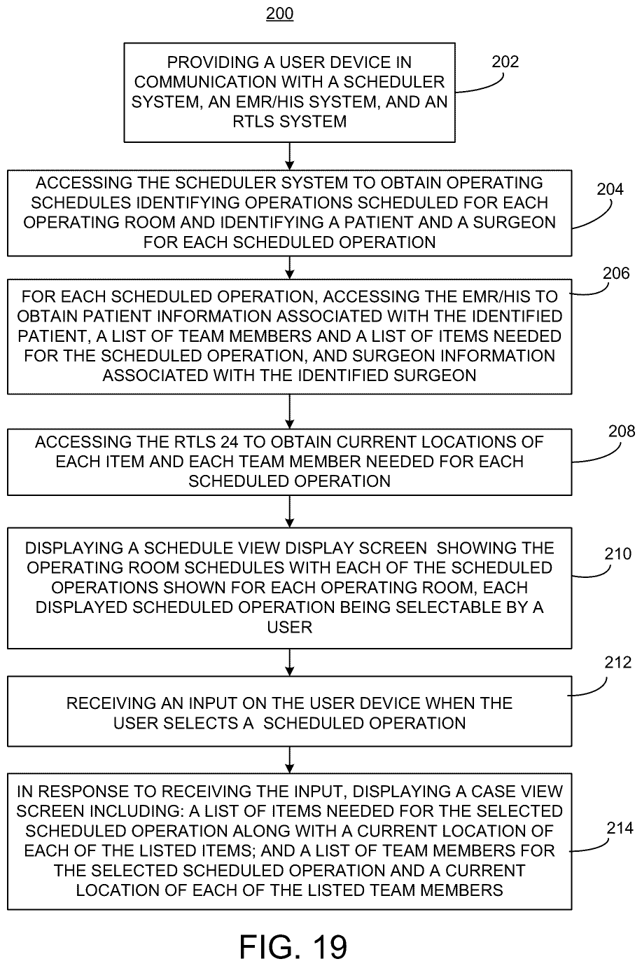

The method for gathering and displaying information pertaining to operating room logistics will now be described with reference to FIG. 19. This method is described below as being implemented by the processor 50 using data from the scheduler system 20, the EMR/HIS 22, and the RTLS 24. This method may be a subroutine executed by any processor, and thus this method may be embodied in a non-transitory computer-readable medium having stored thereon software instructions that, when executed by the processor 50, cause the processor 50 to gather and display information pertaining to operating room logistics, by executing the steps of the method described below. In other words, aspects of the inventive method may be achieved by software stored on a non-transitory computer-readable medium or software modifications or updates to existing software residing in a non-transitory computer-readable medium. Such software or software updates may be downloaded into a non-transitory computer-readable media, such as the memory 56 of the user device 14, 16.

FIG. 19 is a flowchart showing an example of a method 200 for gathering and displaying information pertaining to operating room logistics. The method 200 includes the step of providing a user device 14, 16 in communication with a scheduler system 20, an EMR/HIS 22, and an RTLS system 24 (step 202). The method 200 next includes the step of accessing the scheduler system 20 using the user device 14, 16 to obtain operating schedules identifying operations scheduled for each operating room and identifying a patient and a surgeon for each scheduled operation 104_{x,y} (step 204). For each scheduled operation 104_{x,y}, the user device 14, 16 accesses the EMR/HIS 22 to obtain patient information associated with the identified patient, a list of team members 142, and a list of items 132 needed for the scheduled operation, and surgeon information associated with the identified surgeon (step 206). Accessing the RTLS 24 using the user device 14, 16 to obtain current locations 135 of each item 132 and each team member 142 needed for each scheduled operation 104_{x,y} (step 208). The user device 14, 16 then displays a schedule view display screen 100 on the display 52 showing the operating room schedules with each of the scheduled operations 104_{x,y} shown for each operating room, each displayed scheduled operation 104_{x,y} being selectable by a user (step 210). The user device 14, 16 receives an input when the user selects a scheduled operation 104_{x,y} (step 212). In response to receiving the input, the user device 14, 16 displays a case view screen 120 on the display 52 including: a list of items 132 needed for the selected scheduled operation 104_{x,y} along with a current location 135 of each of the listed items 132; and a list of team members 142 for the selected scheduled operation 104_{x,y} and a current location 135 of each of the listed team members 142 (step 214).

The method 200 may further include displaying message and call icons 128 and 127 on the case view display screen 120 for each team member 142 and establishing a communication link with an SMS/call system 27 upon selection of the message icon 128 or call icon 127 to message or call the team member 142. The method 200 may further include filtering the schedule view display screen 100 to display only those scheduled operations 104_{x,y} associated with specified personnel. Further, the method 200 may include displaying a search screen 190 to receive search criteria and to provide a list of items 132 or team members 142 and their locations 135. The method 200 may include displaying status indicators 133 in the case view display screen 120 for each team member 142 and each item 132. The method 200 may also include displaying case notes in the case view display screen 120 and displaying a corresponding note icon 105 in the schedule view display screen 100 to display any case notes in a pop-up case note window 113 when the note icon 105 is selected.

According to one aspect of the present disclosure, an operating room logistics system is provided that includes a user device having a processor and a display, the user device configured to: access a scheduler system to obtain operating schedules identifying operations scheduled for each operating room and identifying a patient and a surgeon for each scheduled operation; for each scheduled operation, access an EMR/HIS to obtain patient information associated with the identified patient, a list of team members and a list of items needed for the scheduled operation, and surgeon information associated with the identified surgeon; access an RTLS to obtain current locations of each item and each team member needed for each scheduled operation; display a schedule view display screen on the display of the user device showing the operating room schedules with each of the scheduled operations shown for each operating room, each displayed scheduled operation being selectable by a user; receive an input on the user device when the user selects a scheduled operation; and in response to receiving the input, display a case view display screen on the display of the user device including: a list of items needed for the selected scheduled operation along with a current location of each of the listed items as retrieved from the RTLS; and a list of team members for the selected scheduled operation and a current location of each of the listed team members as retrieved from the RTLS.

According to another aspect of the present disclosure, the surgeon information includes surgeon preferences, and the user device is further configured to display the surgeon preferences in the case view display screen.

According to another aspect of the present disclosure, surgeon preferences includes any one or more of the following: position preference, specific equipment preferences, preparation preferences, local anesthesia preferences, and notes as to preferences.

According to still another aspect of the present disclosure, the user device is further configured to display a message icon and a call icon on the case view display screen for each listed team member and to establish a communication link with an SMS/call system upon selection of the message icon or call icon to message or call the listed team member.

According to yet another aspect of the present disclosure, the user device is further configured to filter the schedule view display screen to display only those scheduled operations associated with specified personnel.

According to still another aspect of the present disclosure, the user device is further configured to display a search display screen to receive search criteria and to provide a list of items or team members and their locations.

According to yet another aspect of the present disclosure, the user device is further configured to display status indicators in the case view display screen for each listed team member and each listed item.

According to still another aspect of the present disclosure, the user device is further configured to display case notes in the case view display screen, and to display a corresponding note icon in the schedule view display screen to display any case notes in a pop-up case note window when the note icon is selected.

According to yet another aspect of the present disclosure, the user device is further configured to access the RTLS periodically at a rate that appears to a user as nearly continuously updating the locations of the items and the team members.

According to yet another aspect of the present disclosure, the user device is further configured to determine if a scheduled operation is behind schedule and generates a status indicator on the schedule view display screen indicating that the scheduled operation is behind schedule.

According to yet another aspect of the present disclosure, the user device is further configured to display start time and the elapsed time on the case view display screen.

According to another aspect of the present disclosure, a method is provided of gathering and displaying information pertaining to operating room logistics, the method including: providing a user device in communication with a scheduler system, an EMR/HIS system, and an RTLS system; accessing the scheduler system using the user device to obtain operating schedules identifying operations scheduled for each operating room and identifying a patient and a surgeon for each scheduled operation; for each scheduled operation, accessing the EMR/HIS using the user device to obtain patient information associated with the identified patient, a list of team members and a list of items needed for the scheduled operation, and surgeon information associated with the identified surgeon; accessing the RTLS using the user device to obtain current locations of each item and each team member needed for each scheduled operation; displaying a schedule view display screen on the user device showing the operating room schedules with each of the scheduled operations shown for each operating room, each displayed scheduled operation being selectable by a user; receiving an input on the user device when the user selects a scheduled operation; and in response to receiving the input, displaying a case view display screen on the user device including: a list of items needed for the selected scheduled operation along with a current location of each of the listed items; and a list of team members for the selected scheduled operation and a current location of each of the listed team members.

According to another aspect of the present disclosure, the surgeon information includes surgeon preferences, and the method further includes displaying the surgeon preferences in the case view display screen.

According to still another aspect of the present disclosure, the method further includes displaying a message icon and a call icon on the case view display screen for each listed team member and establishing a communication link with an SMS/call system upon selection of the message icon or call icon to message or call the listed team member.

According to still another aspect of the present disclosure, the method further includes filtering the schedule view display screen to display only those scheduled operations associated with specified personnel.

According to yet another aspect of the present disclosure, the method further includes displaying a search display screen to receive search criteria and to provide a list of items or team members and their locations.

According to still another aspect of the present disclosure, the method further includes displaying status indicators in the case view display screen for each listed team member and each listed item.

According to yet another aspect of the present disclosure, the method further includes displaying case notes in the case view display screen, and displaying a corresponding note icon in the schedule view display screen to display any case notes in a pop-up case note window when the note icon is selected.

According to yet another aspect of the present disclosure, the method further including determining if a scheduled operation is behind schedule and generating a status indicator on the schedule view display screen indicating that the scheduled operation is behind schedule.

According to yet another aspect of the present disclosure, the method further including displaying start time and the elapsed time on the case view display screen.

According to another aspect of the present disclosure, a non-transitory computer-readable medium is provided having stored thereon instructions that, when executed by a processor in a user device, cause the user device to: access a scheduler system to obtain operating schedules identifying operations scheduled for each operating room and identifying a patient and a surgeon for each scheduled operation; for each scheduled operation, access an EMR/HIS to obtain patient information associated with the identified patient, a list of team members and a list of items needed for the scheduled operation, and surgeon information associated with the identified surgeon; access an RTLS to obtain current locations of each item and each team member needed for each scheduled operation; display a schedule view display screen on the user device showing the operating room schedules with each of the scheduled operations shown for each operating room, each displayed scheduled operation being selectable by a user; receive an input on the user device when the user selects a scheduled operation; and in response to receiving the input, display a case view display screen on the user device including: a list of items needed for the selected scheduled operation along with a current location of each of the listed items; and a list of team members for the selected scheduled operation and a current location of each of the listed team members.

According to another aspect of the present disclosure, the surgeon information includes surgeon preferences, and the instructions further cause the user device to display the surgeon preferences in the case view display screen.

According to still another aspect of the present disclosure, the instructions further cause the user device to display a message icon and a call icon on the case view display screen for each listed team member and to establish a communication link with an SMS/call system upon selection of the message icon or call icon to message or call the listed team member.

According to yet another aspect of the present disclosure, the instructions further cause the user device to filter the schedule view display screen to display only those scheduled operations associated with specified personnel.

According to still another aspect of the present disclosure, the instructions further cause the user device to display a search display screen to receive search criteria and to provide a list of items or team members and their locations.

According to yet another aspect of the present disclosure, the instructions further cause the user device to determine if a scheduled operation is behind schedule and generates a status indicator on the schedule view display screen indicating that the scheduled operation is behind schedule.

According to yet another aspect of the present disclosure, the instructions further cause the user device to display start time and the elapsed time on the case view display screen.

Although the user devices 14, 16 are described as having a “processor”, it will be appreciated that the embodiments described herein may be comprised of one or more conventional processors and unique stored program instructions that control one or more processors to implement, in conjunction with certain non-processor circuits, some, most, or all of the functions of the operating room logistics system 12, as described herein. The non-processor circuits may include, but are not limited to, signal drivers, clock circuits, power source circuits, and/or user input devices. As such, these functions may be interpreted as steps of a method used for operating room logistics. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used. Thus, the methods and means for these functions have been described herein. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

It will be understood by one having ordinary skill in the art that construction of the described disclosure and other components is not limited to any specific material. Other exemplary embodiments of the disclosure disclosed herein may be formed from a wide variety of materials, unless described otherwise herein.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the disclosure, as shown in the exemplary embodiments, is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts, or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present disclosure. The exemplary structures and processes disclosed herein are for illustrative purposes and are not to be construed as limiting.

What is claimed is:

1. An operating room logistics system comprising:
a user device comprising a processor and a display, the user device configured to:
access a scheduler system to obtain operating schedules identifying operations scheduled for each operating room and identifying a patient and a surgeon for each scheduled operation;
for each scheduled operation, access an EMR/HIS to obtain patient information associated with the identified patient, a list of team members and a list of items needed for the scheduled operation, and surgeon information associated with the identified surgeon;
accessing an RTLS to obtain current locations of each item and each team member needed for each scheduled operation;
display a schedule view display screen on the display of the user device showing the operating room schedules with each of the scheduled operations shown for each operating room, each displayed scheduled operation being selectable by a user;
receive an input on the user device when the user selects a scheduled operation; and
in response to receiving the input, display a case view display screen on the display of the user device including:
a list of items needed for the selected scheduled operation along with a current location of each of the listed items as retrieved from the RTLS; and
a list of team members for the selected scheduled operation and a current location of each of the listed team members as retrieved from the RTLS.

2. The operating room logistics system of claim 1, wherein the surgeon information includes surgeon preferences, and the user device is further configured to display the surgeon preferences in the case view display screen.

3. The operating room logistics system of claim 2, wherein surgeon preferences include any one or more of the following: position preference, specific equipment preferences, preparation preferences, local anesthesia preferences, and notes as to preferences.

4. The operating room logistics system of claim 1, wherein the user device is further configured to display a message icon and a call icon on the case view display screen for each listed team member and to establish a communication link with an SMS/call system upon selection of the message icon or call icon to message or call the listed team member.

5. The operating room logistics system of claim 1, wherein the user device is further configured to filter the schedule view display screen to display only those scheduled operations associated with specified personnel.

6. The operating room logistics system of claim 1, wherein the user device is further configured to display a search display screen to receive search criteria and to provide a list of items or team members and their locations.

7. The operating room logistics system of claim 1, wherein the user device is further configured to display status indicators in the case view display screen for each listed team member and each listed item.

8. The operating room logistics system of claim 1, wherein the user device is further configured to display case notes in the case view display screen, and to display a corresponding note icon in the schedule view display screen to display any case notes in a pop-up case note window when the note icon is selected.

9. The operating room logistics system of claim 1, wherein the user device is further configured to determine if a scheduled operation is behind schedule and generates a status indicator on the schedule view display screen indicating that the scheduled operation is behind schedule.

10. A method of gathering and displaying information pertaining to operating room logistics, the method comprising:
providing a user device in communication with a scheduler system, an EMR/HIS system, and an RTLS system;
accessing the scheduler system using the user device to obtain operating schedules identifying operations scheduled for each operating room and identifying a patient and a surgeon for each scheduled operation;
for each scheduled operation, accessing the EMR/HIS using the user device to obtain patient information associated with the identified patient, a list of team members and a list of items needed for the scheduled operation, and surgeon information associated with the identified surgeon;
accessing the RTLS using the user device to obtain current locations of each item and each team member needed for each scheduled operation;
displaying a schedule view display screen on the user device showing the operating room schedules with each of the scheduled operations shown for each operating room, each displayed scheduled operation being selectable by a user;
receiving an input on the user device when the user selects a scheduled operation; and
in response to receiving the input, displaying a case view display screen on the user device including:
a list of items needed for the selected scheduled operation along with a current location of each of the listed items; and
a list of team members for the selected scheduled operation and a current location of each of the listed team members.

11. The method of claim 10 and further including displaying a message icon and a call icon on the case view display screen for each listed team member and establishing a communication link with an SMS/call system upon selection of the message icon or call icon to message or call the listed team member.

12. The method of claim 10 and further including filtering the schedule view display screen to display only those scheduled operations associated with specified personnel.

13. The method of claim 10 and further including displaying a search display screen to receive search criteria and to provide a list of items or team members and their locations.

14. The method of claim 10 and further including displaying status indicators in the case view display screen for each listed team member and each listed item.

15. The method of claim 10 and further including determining if a scheduled operation is behind schedule and generating a status indicator on the schedule view display screen indicating that the scheduled operation is behind schedule.

16. A non-transitory computer-readable medium having stored thereon instructions that, when executed by a processor in a user device, cause the user device to:
access a scheduler system to obtain operating schedules identifying operations scheduled for each operating room and identifying a patient and a surgeon for each scheduled operation;
for each scheduled operation, access an EMR/HIS to obtain patient information associated with the identified patient, a list of team members and a list of items needed for the scheduled operation, and surgeon information associated with the identified surgeon;
accessing a RTLS to obtain current locations of each item and each team member needed for each scheduled operation;
display a schedule view display screen on the user device showing the operating room schedules with each of the scheduled operations shown for each operating room, each displayed scheduled operation being selectable by a user;
receive an input on the user device when the user selects a scheduled operation; and
in response to receiving the input, display a case view display screen on the user device including:
a list of items needed for the selected scheduled operation along with a current location of each of the listed items; and
a list of team members for the selected scheduled operation and a current location of each of the listed team members.

17. The non-transitory computer-readable medium of claim 16, wherein the instructions further cause the user device to determine if a scheduled operation is behind schedule and generates a status indicator on the schedule view display screen indicating that the scheduled operation is behind schedule.

18. The non-transitory computer-readable medium of claim 16, wherein the instructions further cause the user device to display a message icon and a call icon on the case view display screen for each listed team member and to establish a communication link with an SMS/call system upon selection of the message icon or call icon to message or call the listed team member.

19. The non-transitory computer-readable medium of claim 16, wherein the instructions further cause the user device to filter the schedule view display screen to display only those scheduled operations associated with specified personnel.

20. The non-transitory computer-readable medium of claim 16, wherein the instructions further cause the user device to display a search display screen to receive search criteria and to provide a list of items or team members and their locations.
